(19) United States
(12) Patent Application Publication
(10) Pub. No.: US 2024/0268713 A1
(43) Pub. Date: Aug. 15, 2024

(54) System and Methods for Analyzing User Adherence During Guided Breathing

(71) Applicant: Apple Inc., Cupertino, CA (US)

(72) Inventors: Thilaka S. Sumanaweera, San Jose, CA (US); Anirban Chatterjee, Sunnyvale, CA (US); Gopal Valsan, Gilroy, CA (US)

(21) Appl. No.: 18/422,923

(22) Filed: Jan. 25, 2024

Related U.S. Application Data
(60) Provisional application No. 63/444,728, filed on Feb. 10, 2023.

(51) Int. Cl. A61B 5/113; A61B 5/00; A61B 5/08; A61B 5/107; A61B 5/11

(52) U.S. Cl. CPC A61B 5/1135; A61B 5/0816; A61B 5/1079; A61B 5/1128; A61B 5/7405; A61B 5/742

(57) ABSTRACT

Embodiments include a system for analyzing breathing parameters of a user. The system can include an optical sensing unit configured to detect movement of a torso of the user and output one or more signals indicative of the movement of the torso. The system can also include an electronic device configured to request for the user to breathe according to a breathing profile during a time period. The system can include a processing unit configured to receive the one or more signals generated by the optical sensing unit during the time period and determine an adherence metric using the one or more signals and the breathing profile, where the adherence metric indicating a correspondence between movement of the torso and the breathing profile requested during the time period.

### CROSS-REFERENCE TO RELATED APPLICATION

This application is a nonprovisional of, and claims the benefit under 35 U.S.C. § 119(e) of, U.S. Provisional Patent Application No. 63/444,728, filed Feb. 10, 2023, titled “System and Methods for Analyzing User Adherence During Guided Breathing” the contents of which are incorporated herein by reference.

### FIELD

The described embodiments relate generally to systems and methods for analyzing breathing parameters of a user. More particularly, the present embodiments relate to systems and methods that monitor a user's breathing parameters during guided breathing activities.

### BACKGROUND

Individuals may use an electronic device to perform and track health related activities. For example, a user may perform a guided breathing exercise, meditation exercise or other activity to reduce stress. In some cases, a guided breathing or meditation exercise can include listening to and/or watching instructions on an electronic device. A user may try to perform the instructed actions but may not know whether or how well the actions they are performing match the instructed actions. It may be desirable to have a way to track a user's movement during health related activities such as guided breathing.

### SUMMARY

Embodiments are directed to a system for analyzing breathing parameter of a user that includes an optical sensing unit that is configured to detect movement of a torso of the user and output one or more signals indicative of the movement of the torso. The system can also include an electronic device that is configured to request for the user to breathe according to a breathing profile during a time period. The system can include a processing unit that is configured to receive the one or more signals output by the optical sensing unit during the time period and determine an adherence metric using the one or more signals and the breathing profile, where the adherence metric indicates a correspondence between movement of the torso and the breathing profile requested during the time period.

Embodiments are also directed to a system for analyzing user adherence to a requested breathing profile. The system can include a sensing unit configured to detect movement of a torso of a user and output one or more signals indicative of the detected movement of the torso. The system can include an electronic device configured to provide a request for a user to breathe according to a breathing profile during a time period. They system can also include a processing unit programmed to receive the one or more signals and determine an adherence metric using the one or more signals and the first breathing profile.

Embodiments are further directed to a method for measuring breathing parameter of a user. The method can include outputting a request for the user to breath in accordance with a breathing profile during a time period from an electronic device. The method can include obtaining, by an optical sensing unit, a set of respiratory measurements during the time period, the set of respiratory measurements comprising measurements corresponding to movement of a torso of the user. The method can also include determining, by a processing unit, an adherence metric for the user using the set of respiratory measurements and the breathing profile, where the adherence metric indicates a correspondence between movement of the torso and the breathing profile requested during the time period.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows an example system for determining respiratory function of a user;

FIG. 2 shows an example process for measure respiratory movements of a user;

FIGS. 3A and 3B show an example chest movement analysis for determining breathing parameters of a user;

FIG. 4 shows an example signal flow between different devices of a respiratory monitoring system;

FIG. 5 shows an example process for performing an enrollment session for respiratory sensing;

FIG. 6 shows an example process for performing a respiratory sensing session for a user;

FIGS. 7A and 7B show examples of torso movement data and adherence data;

FIG. 8 shows an example of a breathing profile and torso movement data collected for the breathing profile;

FIG. 9 shows an example process for collecting date from multiple regions of the torso during a breathing exercise;

FIG. 10 shows an example torso movement data for multiple regions of the torso during a breathing exercise; and

FIG. 11 is an example block diagram of a respiratory monitoring system.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments disclosed herein are directed to systems and methods for monitoring respiratory movements of a user during a guided breathing session. The systems and methods can include an electronic device that instructs a user to breathe according to a defined breathing profile. The breathing profile can define one or more breathing parameters that the user is intended to perform.

As used herein, a “breathing profile” includes one or more target breathing parameters that the user will try to achieve during breathing. For example, a breathing profile may set an overall target breathing rate (e.g., a specified number of breaths per minute) and/or a target breathing depth (e.g., how fully a user should inhale and/or exhale). Additionally or alternatively, the breathing profile may set one or more additional target characteristics for a user's breathing.

In some instance, the breathing profile may set a target breathing pattern, which represents the relative amounts of time a user is inhaling, exhaling, or holding their breath during a given breath. One example of a breathing profile includes box breathing, in which, for a given breath, the user inhales for a first amount of time, holds their breath for a second amount of time, exhales for a third amount of time, and holds their breath for a fourth amount of time. Another example of a breathing profile includes timed breathing exercise (e.g., 4-7-8 breathing), in which, for a given breath, the user inhales for a first amount of time (e.g., four seconds), holds their breath for a second amount of time (e.g. seven seconds), and exhales for a third amount of time (e.g., eight seconds).

Additionally or alternatively, the breathing profile may set a target breathing style, which dictates how a user is supposed to move their body during breathing. For example, a breathing style may include diaphragmatic breathing (also called “belly breathing”) in which a user focuses on moving their abdomen while breathing. Additionally or alternatively, a breathing style may include pursed lip breathing, in which a user purses their lips to control the flow of air during inhalation and/or exhalation. A breathing style may also include inhalation through the nose, and exhalation through the mouth. A breathing style may include inhalation through one nostril in the nose, and exhalation through the other nostril with a finger closing the nostril not in use. Additionally or alternatively, a breathing style may include specific types of poses, such as sitting down, standing up, lying down.

The system and methods can include measuring breathing parameters of a user while the user performs a guided breathing exercise. The measurements taken during the guided breathing exercise can be analyzed to determine one or more respiratory metrics for the user. In some cases, the system can include an electronic device that outputs instructions for performing the guided breathing exercise. The system can also include one or more sensors that measure movement of a user's torso during the guided breathing exercise. For example, the guided breathing exercise can include instructing a user to breathe according to a breathing profile and measuring the user's torso movement while the breathing profile is being instructed. As used herein the term “torso” is intended to include portions of the user's upper body that moves as part of the breathing motion of the user. Accordingly, the term torso can include, for example, a portion of the user's abdomen, chest, and/or back.

The system and methods can include analyzing the movement of the user's torso and the requested breathing profile to determine one or more adherence metrics. The one or more adherence metrics can characterize a correspondence between breathing movements of the user and the requested breathing profile. For example, the adherence metric may indicate how closely a user is matching one or more target breathing parameters (e.g., a target breathing rate, a target breathing depth, etc.) of the breathing profile. In some cases, multiple adherence metrics can be used to characterize different breathing parameters. For example, a first adherence metric may indicate how closely a user is matching a requested breathing rate and a second adherence metric may indicate how closely a user is matching a requested breathing depth. In some cases, a single adherence metric may characterize multiple breathing parameters. (e.g., breathing rate and breathing depth). The adherence metric may be a time-varying signal that represents the level of correspondence to the requested breathing profile over time.

The systems described herein provide one or more outputs to the user based on the breathing profile. In some cases, the system may use auditory outputs as instructions for a particular breathing profile. The auditory outputs may include cues (e.g., verbal cues, simulated or recorded breathing sounds, etc.) that indicated when a user should start and stop inhaling, start and stop exhaling, hold their breath and so on. In some cases, the system may use one or more determined adherence metrics to provide and/or adjust the instructional outputs. For example, an adherence metric may indicate that the user is breathing slower than instructed. The system may output an instruction for the user to breathe at a quicker rate, and or modify the outputs (e.g., a simulated breathing sound) to emphasize the breathing rate. Additionally or alternatively, the system may use visual outputs and/or haptic outputs. The visual and/or haptic outputs can include graphics that provide visual cues based on the requested breathing profile and/or the measured torso movements of the user. Some visual (and/or haptic) cues may be used to indicate the requested breathing profile and other visual (and/or haptic) cues may be used to indicate how closely a user is matching the requested breathing profile. visual cues may include expanding and contracting shapes, color changes or any other suitable graphics. The system can include an electronic device that provides an audio, visual, haptic, or other suitable output or combinations of outputs to a user.

In some cases, the system can provide guided breathing based on one or more environmental conditions of the user. For example, the guided breathing protocol and/or when a guided breathing session is performed can take into account factors such as air quality, temperature, or other environmental parameters; user parameters such as heart rate, breathing rate, stress, perspiration; timing and/or location parameters (e.g., based on GPS data); or other suitable parameters.

In some cases, the system can include an electronic device that outputs instructions for the guided breathing exercise and includes a depth sensor for measuring torso movement of a user. The depth sensor may generate a depth map including calculated distances, some or all of which may be used in the various techniques described below. The depth information may be calculated in any suitable manner. In one non-limiting example, a depth sensor may utilize stereo imaging, in which two images are taken from different positions, and the distance (disparity) between corresponding pixels in the two images may be used to calculate depth information. In another example, a depth sensor may utilize structured light imaging, whereby the depth sensor may image a scene while projecting a known pattern (typically using infrared illumination) toward the scene, and then may look at how the pattern is distorted by the scene to calculate depth information. In still another example, a depth sensor may utilize time of flight sensing, which calculates depth based on the amount of time it takes for light (typically infrared) emitted from the depth sensor to return from the scene. A time-of-flight depth sensor may utilize direct time of flight or indirect time of flight and may illuminate the entire field of coverage at one time or may only illuminate a subset of the field of coverage at a given time (e.g., via one or more spots, stripes, or other patterns that may either be fixed or may be scanned across the field of coverage). Additionally or alternatively, one or more cameras may captures images (e.g., RGB images, infrared images, or the like) and analyze these images as part of measuring torso movement. For example, optical flow processing of videos (e.g., RGB videos and/or infrared videos), and/or any other suitable imaging techniques can be used to measure torso movement and/or otherwise extract respiratory metrics.

The electronic device can include a display, speakers, one or more microphones, and haptic output devices one or more of which may be used to present the guided breathing exercise to the user. In some embodiments the electronic device includes an optical sensing unit that can measure torso movements of the user. For example, the optical sensing unit can include a depth sensor that measures changes in depth of the torso of the user. These depth measurements can be used to determine breathing parameters such as an adherence metric to a requested breathing profile. Example electronic devices can include smartphones, tablets, smartwatches, or any other suitable electronic devices. In some cases, a first electronic device can output instructions for a guided breathing exercise and a second electronic device can measure chest movements of the user.

Additionally or alternatively, the system can include a motion tracking sensor that measures torso movements of a user. For example, the motion tracking sensor can be part of an electronic device and the electronic device can be placed on or otherwise coupled to a torso of a user (for the purpose of this application, an object is considered to be “coupled” to a torso of the user while it is held in a fixed relationship to the torso). The electronic device may be held in place relative to the torso of a user during a measurement such that the electronic device moves in unison with the chest portion. In some instances, a user may hold the electronic device in place against the chest (e.g., using their hand, or by placing their wrist against their chest while wearing a smartwatch). In other instances, the electronic device may be temporarily affixed to a user's torso (e.g., using a strap, fastener, adhesive, or the like). Accordingly, the electronic device may move with the user's torso and measure these torso motions. In some cases, the motion tracking sensor can include one or more accelerometers, gyrometers, wireless positioning systems, or other suitable sensors. The chest movement measured by the one or more motion tracking sensors can be used to determine breathing parameters for the user, which can include breathing power, depth signal morphology, or other suitable parameters such as those described herein.

In some cases, the system can utilize multiple electronic devices to measure breathing parameters for a user. For example, the system can include a first electronic device that has an optical sensing unit for measuring chest movement as described herein. The system can also include a second electronic device that includes a motion tracking sensor. For example, the first electronic device can be a smartphone or a tablet and the second electronic device can be a smartwatch. In some cases, wireless positioning can be used to track motion of a user's chest. For example, each of the electronic devices can include one or more antennas, and wireless signals transmission (e.g., radio-frequency, ultra-wide band signals, and so on) can be used to determine distances and/or orientation of the electronic devices with respect to each other. A first electronic device can be positioned at a distance from the user and the second electronic device can be coupled to the chest of the user, and the changes in distance between the devices can be used to determine chest movement of the user. This wireless positioning data may be used in addition to or as alternative to optical depth measurements, motion tracking, or other suitable processes. Additionally or alternatively, the system may use imaging and/or depth sensing functionality to identify and/or measure a distance between the first and second electronic devices.

Embodiments can include performing an enrollment period to determine one or more baseline data sets for a user, which may be used to correlate torso movement to specific breathing parameters such as torso movement corresponding to a user's maximum inhale or exhale conditions. Enrollment processes can include analyzing measurement conditions to determine whether suitable measurements can be made. For example, in some cases the clothing worn by a user may prevent the system from being able to detect sufficient torso movement. The enrollment process can include analyzing a user's clothing to determine whether the sensing systems can accurately measure respiration parameters, such as torso movements of the user. In some cases, the enrollment period can include measuring a user's normal breathing patterns and/or requesting a user to breathe at different breathing rates and measuring respiration parameters at the requested breathing rates. In some cases, the enrollment period can include measuring breathing parameters using traditional analysis methods such as spirometry measurements. The spirometry data can be compared and/or correlated to measurements taken by the respiratory sensing system described herein. Additionally or alternatively, the enrollment period can include analyzing a user's breathing parameters at different conditions such as standing positions, sitting positions, postures, mouth shapes, monitoring breathing sounds and so on.

The data obtained during the enrollment period can be used to generate one or more user models. In some cases, the user model can be a parameterized model and enrollment data and/or data from respiratory sensing sessions can be used to generate a parameterized torso and/or body model. The parameterized model can include data related to a user's shape, pose, body composition, height, weight, and/or other demographic data. In some cases, the parameterized model can be generated using one or more images of a user, which may include images from various perspectives such as front, side, and/or back images of a user.

In some cases, a guided breathing session can be, with a user's permission, prompted or initiated by a remote user, such as caregiver, doctor, or other healthcare provider. For example, a guided breathing session may be performed, scheduled (i.e., for the user to perform later), or otherwise may be made available as part of a telehealth service. In some instances, while a user is engaged with a healthcare provider during a telemedicine session, the healthcare provided may prompt a user to perform a guided breathing session during the telemedicine session. Alternatively, the healthcare provide may assign a particular guided breathing session for the user to perform at a different time.

When a guided breathing session is prompted or initiated by a remote user, the user's device may receive a target breathing profile (as well as any other information regarding the guided breathing session) that is transmitted from a device or server associated with the remote user. The user device may use the received target breathing profile during a guided breathing session in any suitable manner as described herein to determine adherence. In some instances, with the user's permission, information about the adherence (e.g., adherence data, adherence metrics) or other information associated with the guided breathing session may be transmitted to the remote user.

In some cases, the user's device may be configured to derive the target breathing profile based on information received during a telemedicine session. For example, during a telemedicine session, the remote user may transmit, for example audio, visual or other outputs that are presented to the user during the telemedicine session, which may prompt a user to breath according to a certain breathing profile. In these cases, the user device may receive and analyze the outputs (e.g., audio signals) and generate the target breathing profile that is being transmitted to the user's device. The target breathing profile may then be used to measure adherence as described herein.

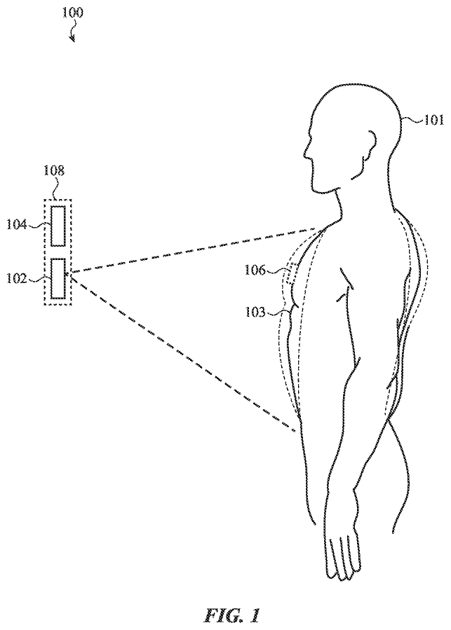

These and other embodiments are discussed below with reference to FIGS. 1-11. However, those skilled in the art will readily appreciate that the detailed description given herein with respect to these Figures is for explanatory purposes only and should not be construed as limiting.

FIG. 1 shows an example system 100 for measuring breathing parameters of a user 101. The system 100 can include a sensing unit 102 that is configured to measure one or more parameters of the user 101 and an output unit 104 that is configured to provide outputs to the user 101. In some embodiments that system 100 can include a second device 106 that contacts the user's 101 torso 103 and is configured to measure parameters of the user 101. The sensing unit 102, the output unit 104 and the second device 106 can be communicably coupled and may be operated in coordination to determine parameters of the user 101, such as one or more respiration parameters as described herein.

In some cases, the sensing unit 102 and the output unit 104 can be integrated into an electronic device 108 such as a smartphone, tablet, digital media player (e.g., mp3 player), smartwatch, laptop computer, desktop computer, smart speaker, furniture objects such as a fitness mirror, bed sensing devices or other electronic devices. The electronic device 108 may include a housing and a transparent cover (which may be referred to simply as a “cover”) coupled with the housing and positioned over a display. The cover and the housing along with other components may form a sealed internal volume of the electronic device, which may contain the internal electrical components of the electronic device. In some cases, the cover defines substantially the entire front face and/or front surface of the electronic device 108. The cover may also define an input surface. For example, as described herein, the electronic device 108 may include touch and/or force sensors that detect inputs applied to the cover. The cover may be formed from or include glass, sapphire, a polymer, a dielectric, or any other suitable material.

The output unit 104 can include a display that is positioned under the cover and at least partially within the housing. The display may define an output region in which graphical outputs are displayed. Graphical outputs may include graphical user interfaces, user interface elements (e.g., buttons, sliders, etc.), text, lists, photographs, images, videos, or the like. The display may include a liquid-crystal display (LCD), an organic light emitting diode display (OLED), or any other suitable components or display technology. In some cases, the display may output a graphical user interface with one or more graphical objects that display information.

The display may be touch- and/or force-sensitive and include or be associated with touch sensors and/or force sensors that extend along the output region of the display and which may use any suitable sensing elements and/or sensing techniques. Using touch sensors, the electronic device 108 may detect touch inputs applied to the cover, including detecting locations of touch inputs, motions of touch inputs (e.g., the speed, direction, or other parameters of a gesture applied to the cover), or the like. Using force sensors, the electronic device 108 may detect amounts or magnitudes of force associated with touch events applied to the cover. The touch and/or force sensors may detect various types of user inputs to control or modify the operation of the device, including taps, swipes, multiple finger inputs, single- or multiple-finger touch gestures, presses, and the like.

Additionally or alternatively, the output unit 104 can include one or more speakers, which can be integrated with the housing of the electronic device 108. The speakers can be configured to provide audio outputs to the user 101, which can include instructions for guided breathing exercises, other user feedback and so on as described herein. The speakers can be part of the electronic device and/or integrated with other devices that are separated from the electronic device. For example, the output unit 104 may include one or more earbuds or headphones that are worn by the user and communicatively coupled with the electronic device.

In some cases, the output unit 104 can also include one or more haptic actuators that provide tactile outputs to the user 101. The haptic actuators can be part of the electronic device 108 and/or integrated on devices that are separate from the electronic device. For example, the electronic device 108 can include a smartphone that has an optical sensing unit 102 and the output unit 104 can include one or more haptic actuators that are integrated with a wearable device such as a smartwatch. In this regard, the output unit 104 can include different components that are integrated with different devices and communicably coupled to provide coordinated outputs. For example, the display on the electronic device 108 may provide a visual cue for a guided breathing exercise, the speakers (integrated with the electronic device or other device such as headphones) can provide an audio cue that is coordinated with the visual cue and/or the haptic actuators (e.g., located on a smartwatch) may provide a haptic cue that is coordinated with the other outputs.

In some cases, the output unit 104 can be associated with a first electronic device and the sensing unit 102 can be associated with a different electronic device. The output unit 104 and the sensing unit 102 can coordinate respiratory measurements, which can include transmitting one or more signals between the sensing unit 102 and the output unit 104. In cases where the sensing unit 102 and the output unit 104 are associated with different electronic devices, signals can be transmitted between the different devices via a suitable wireless transmission protocol. The signals can indicate when the output unit 104 is outputting a request for a user to breathe according to a breathing profile. Additionally or alternatively, the signals can include a time period, duration and/or end associated with the request. Accordingly, the sensing unit 102 can use these signals to associate measurement data with the requested breathing profile and/or a time period associated with the breathing profile.

The sensing unit 102 can include an optical sensing unit that measures movement of the user 101. The optical sensing unit can include a depth measurement sensor (or sensors) that can determine a distance between the user 101 and the sensing unit 102. A depth measurement sensor may include a time-of-flight sensor, a structured light sensor, a stereo camera, or the like. The optical sensing unit 102 can include a camera or other suitable imaging device that is configured to capture an image of a scene (which may in turn be used to identify one or more regions on a user). For example, the optical sensing unit 102 can include a camera that can image the user's body and the optical sensing unit 102 and/or other components of the electronic device 108 (e.g., processor) can be configured to identify anatomical features of the user, such as a torso of a user. The depth sensor and the camera can have overlapping fields of view, such that the identification of anatomical features via the camera can be used by the device to associate anatomical features with measurements made by the depth sensor.

The electronic device 108 can include additional sensors such as accelerometers, gyrometers, positioning sensors such as global position system (GPS) sensors, wireless positioning systems, altimeters, pressure sensing systems and/or the like. Additionally or alternatively, the electronic device 108 can include physiological sensors such as temperature sensors, heart rate monitors and/or other suitable sensors.

The device 106 can be a wearable electronic device, such as a smartwatch, or other suitable device. In some cases, the device 106 can contact the user's 101 torso by the user holding the device 106 against their torso. In other cases, the device 106 can be coupled to the user's torso, for example by a coupling device. Accordingly, the device 106 may move with the user's 101 torso as they breathe or perform other actions. The device 106 can be communicably coupled to the electronic device 108. The system 100 can measure movement of the user's 101 torso by tracking movement of the second device. For example, the electronic device 108 and the device 106 can each include one or more antennas. The electronic device 108 and the device 106 can transmit signals, such as UWB signals, that can be used to determine distance and/or positions of the device with respect to each other. Accordingly, the electronic device 108 and the device 106 can track movement such as changes in depth of the user's 101 torso using wireless based distance and position sensing. Additionally or alternatively, the device 106 can measure movement of the user's 101 torso based on one or more onboard sensors such as accelerometers, gyrometers, or other suitable position sensors. Additionally or alternatively, a camera, distance sensor or other sensor from the sensing unit 102 can be used to identify the device 106 and/or track movement of the device 106 while it is positioned on the user's 101 torso 103. In some cases, the device 106 may include a display that displays a predetermined image which can be used by the sensing unit 102 to identify and/or track the device 106.

In some cases, the device 106 can include a microphone and/or speaker that can measure sounds of a user (e.g., breathing sounds) and output audio to a user (e.g., audio cues for a guided breathing profile).

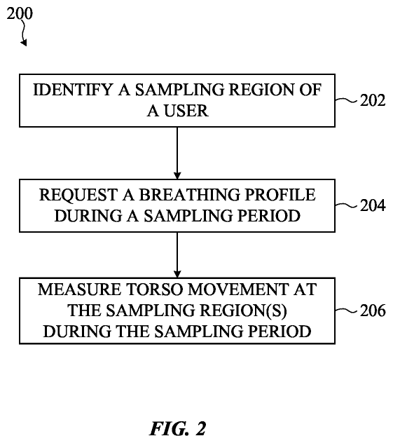

FIG. 2 shows an example process 200 for determining respiratory movements of a user. The process can be performed by the respiratory sensing systems described herein such as a system 100.

At 202, the process 200 can include identifying one or more sampling regions along a user for measuring breathing parameters. The sampling regions can be located across a torso of a user, which can include a chest and/or abdomen of the user. In some cases, the sensing unit can include an optical sensing unit that has one or more cameras that image a torso of the user and identify anatomical features such as a profile of the user's torso, location and/or movement of the user's shoulders, and so on. The sampling regions can be defined based on the identified anatomical features. For example, the system can define multiple sampling regions at different locations along the user's chest and/or abdomen. In some cases, the system may select a sampling region based on the identified anatomical features. The system may measure movement at each of the sampling regions. For example, a camera may be used to identify anatomical features of the user, which are used to define one or more sampling regions. A depth sensor can measure changes in depth of the torso at each of the sampling regions. In some cases, a parameterized body model of the user can be fit to the measurement data to separate breathing induced movements from other movements.

In some cases, the system may select one or more of the sampling regions for generating breathing parameters of a user. For example, the system may compare signal strength, accuracy, measurement stability, and/or the like at each sampling region and select one or more sampling regions based on these metrics. For example, the process 200 may include selecting the sampling region that has the greatest signal strength and/or range of depth measurements. The depth measurements at the selected sampling region(s) can be used to determine respiration parameters such as breathing power, as described herein.

Additionally or alternatively, the system may monitor and collect data for each defined sampling region during one or more sampling periods. The operation 202 can include normalizing the measured torso movement data for each sampling region. Accordingly, the system may be able to use measurement data from different sampling regions during one or more of the sampling periods. If the user moves, the optical sensing unit moves, or other changes occur that affect the signal quality at the different sampling regions, the operation 202 can include dynamically selecting a different sampling region or set of sampling regions for determining a respiration parameter. In some cases, the relative movement between the user and the optical sensing unit may be large enough where a new sampling region(s) is identified and a new selection of a sampling region can occur. Accordingly, a respiration parameter may be based on measurement data from different sampling regions.

At 204 the process 200 can include requesting the user to breathe according to a breathing profile during a sampling period. For example, an output device such as a display, speaker and/or haptic device can output instructions for a user to breathe according to the breathing profile. An electronic device can use a speaker to provide audio outputs corresponding to the breathing profile. Additionally or alternatively, the electronic device can use a display to provide visual outputs corresponding to the breathing profile. The outputs can be implemented in a variety of ways including dynamic outputs that indicate inhale and exhale timing, which a user of the system can mimic to achieve the requested breathing rate.

Instructing a user to breath according to a breathing profile can be implemented in a variety of ways as described herein. For example, the system can instruct a user to breathe at a defined breathing rate (e.g., a defined number of breaths per minute), at a defined breathing depth (e.g., defined in terms of a user's maximum inhale and exhale capacity), and/or other suitable breathing parameters. In some cases, the breathing profile can include a breathing pattern for the user to match, and the system can output instructions that provide breathing cues for the user (e.g., audio outputs, visual outputs, haptics outputs, etc.). For example, the breathing pattern can include a box breathing technique (e.g., four-square breathing), diaphragmatic breathing (e.g., belly breathing), pursed lip breathing, 4-7-8 breathing, and/or any other suitable breathing technique and the outputs can include cues that indicate breathing parameters such as how long to inhale, how long to exhale, how long to a hold their breath after an inhale or exhale, and so on.

One or more adherence metrics may be determined as part of any guided breathing session. In some cases, the selection of a breathing profile for a guided breathing session can be dependent on an activity of a user. For example, a guided breathing session may be initiated to focus on mindfulness (e.g., to facilitate mediation, reduce stress, and so on), and a particular breathing profile may be selected for a mindfulness based guided breathing session. In other cases, a guided breathing session may be initiated to control breathing following exercise and a different breathing profile may be selected for the exercise based guided breathing session. In some cases, the breathing profile can be dynamically selected based on one or more user parameters captured before a guided breathing session such as a user's natural breathing rate and/or one or more parameters captured during the guided breathing session such as their heart rate, their current breathing rate, and/or the like. For example, a first breathing profile can be selected if a user's heart rate (which may be determined using another sensor system) is below a threshold and a different breathing profile can be selected if the user's heart rate is above the threshold.

Depending on the configuration of the guided breathing session, aspects of the breathing profile may be configured to change over time. For example, during a given breathing session, a target breathing rate and/or target breathing depth may change over time. For example, the breathing profile can change according to a defined protocol such as a constant increase in the requested breathing rate (e.g., ramp protocol) and/or increase followed by decreases in the requested breathing rate (e.g., cyclic protocol). In any of these cases, the outputs can dynamically change to indicate the desired breathing rate and/or changes to the user. In some cases, the requested breathing profile can be based on current breathing metrics for a user. For example, the system can measure a current breathing rate of the user, and update the requested breathing profile based on the user's currently measured breathing rate. This may be used to adjust the requested breathing profile, for example, in cases where one or more adherence metrics satisfy criteria that indicate a user is deviating too far from the requested breathing profile, the breathing profile can be updated to a slower rate.

In some cases, adjusting the breathing profile during a guided breathing exercise may help a user meet the goals of a particular guided breathing exercise. For example, a guided breathing exercise for mindfulness may be configured to help a user relax and focus on a meditation activity. If one or more adherence metrics determined during the guided breathing exercise indicate that that user is not able to sufficiently match the requested breathing profile (e.g., a determined adherence metric is below a defined threshold), the system may adjust based on the user's abilities. For example, the system may determine a user's inhalation and exhalation capacity. The breathing profile for the guided breathing session may be updated based on the user's inhalation and exhalation capacity. Accordingly, in some cases, the system can collect baseline data for a user to establish breathing parameters for the user (e.g., at a rest state) and update a breathing profile based on the user's specific baseline breathing data.

The system may determine adherence data for different durations during a guided breathing exercise. In some cases, the sampling period for determining one or more adherence metrics can be a defined period of time that captures measurement data at the requested breathing profile. In other cases, the sampling period can be a defined number of breathing cycles, which may vary in time depending on the requested profile and/or the user's actual measured breathing profile. In other examples, the duration of the sampling period can be dynamically adjusted based on measured respiration data for the user. The system can analyze collected data during the sampling period, the results of which can be used to adjust the sampling period. For example, the system may analyze averages, deviations or other metrics associated with the measurement data that is being collected, and adjust the sampling period based on these metrics. For example, if movement of the sensing unit negatively impacts the quality of measurement data, the system may extend the sampling period. In some cases, this can include having the first sampling period run until a defined metric is satisfied, which may include sampling until a deviation between measured respiration cycles satisfies a threshold. In other cases, the system can analyze collected data during the sampling period.

At 206, the process 200 can include measuring movement of one or more sampling regions during the first sampling period. This can include measuring changes in the depth of the chest, which correspond to expansion and contraction of the chest due to respiration. The optical sensing unit can be part of an electronic device, such as a smartphone, and the electronic device can include additional sensors such as accelerometers, gyroscopes, GPS positioning systems, other wireless positioning systems, altimeters, and/or other position sensors. These sensors can monitor motion of the electronic device thereby determining whether the optical sensing unit moves during the first sampling period. In cases where the optical sensing unit is stationary, distance measurement by the optical sensing unit may directly correlate to depth changes of the sampling regions along the user's chest. In cases where the optical sensing unit is moving, measurement data from the position sensors (accelerometers, gyroscopes, etc.) may be used to account for movement of the optical sensing unit and determine depth changes of the sampling regions along the user's chest. Accordingly, the electronic device may be able to determine depth changes when the electronic device is stationary and/or if the electronic device moves or changes orientation during the sampling period.

Additionally or alternatively, the operation 206 can include measuring body movements of a user and compensating for those body movements to determine torso movement. During the sampling period, the user's body (e.g., entire torso) may move relative to the optical sensing unit due to the user changing position, shifting, or making some other large body motion. The optical sensing unit may track these gross body motions to differentiate them from torso movements resulting from breathing. For example, a parameterized body model of a user can be used to differentiate and/or extract breathing induced changes for other measurement data such as gross body motion. In some cases, the optical sensing unit can use one or more cameras to identify anatomical features of the user, which can include identifying a profile of the user's torso, identifying anatomical features such as the user's shoulder, neck, head, abdomen and so on. In some cases, the optical sensing unit can measure distance changes of these different anatomical regions. Accordingly, movement measurements from the one or more sampling regions can be compared to distance measurements of one or more of these anatomical regions, and chest (and/or abdomen) movements can be separated from other motions of the user.

In some cases, the sensing unit can include a position sensing unit that contacts and/or is coupled to one or more sampling regions on the user. The position sensing unit can measure movement of the torso by movement of the device. For example, the movement of the position sensing unit may be tracked using motion sensors (e.g., accelerometers, gyroscopes, and so on) or by measuring relative motion between the position sensing unit and another device, as described herein. The position sensing unit can be in addition to the optical sensing unit or used as an alternative sensing system. For example, the position sensing unit can include a smartwatch that is worn by the user and the user can be requested to place their hand against their chest to measure chest movements via movements of the smartwatch. In other examples, the smartwatch or other position sensing device such as a low-energy near-field tracking device can be coupled to a chest region of the user. For example, a coupling device such as a band, strap, adhesive-based device, or other suitable device may be used to couple the position sensing device to a sampling region of the user. Accordingly, the motion of the position sensing device may correspond to chest movements of the user, which can be used to determine depth changes and/or other movements of the user during the first sampling period.

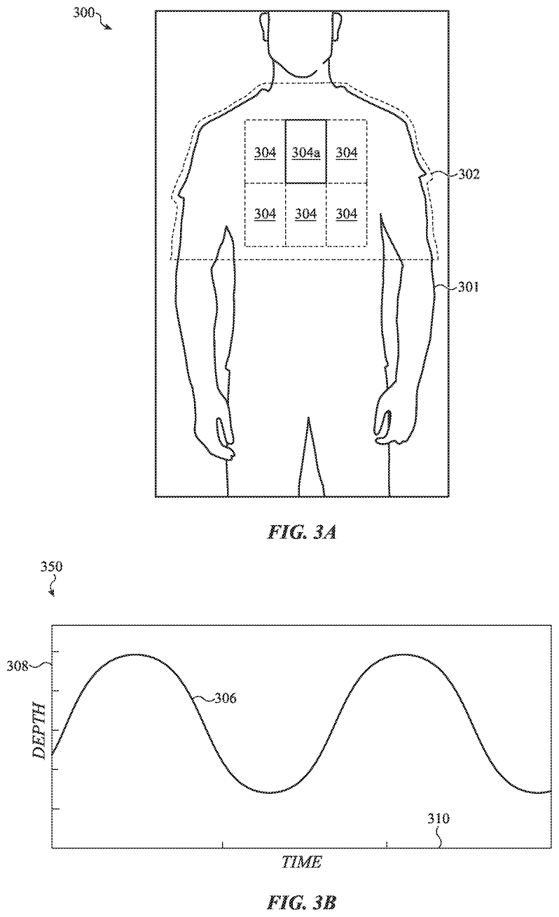

FIGS. 3A and 3B show an example chest movement analysis for determining breathing parameters of a user 301. The example chest movement analysis is illustrated visually in FIGS. 3A and 3B, however, one or more of these processes can be performed by a computer and may not produce and/or display the graphical elements displayed in FIGS. 3A and 3B.

FIG. 3A shows an example of determining one or more sampling regions 304 that are determined from a field of view 300 of one or more of the sensing systems. The one or more sampling regions 304 can be locations for measuring torso movement of the user 301 during a sampling period. An optical sensing unit can identify a torso 302 of the user 301. In some cases, this can include using a camera to capture one or more images of the user 301, which can include capturing a real-time/continuous video feed of the user 301. In some cases, the images captured by the camera can be analyzed in real-time or near-real time to identify changes in the user 301 such as changes in position, posture, orientation, and so on. The analysis can be continually updated to account for these changes captured by the camera. The camera may be capable of capturing color images, black and white images, and/or non-visible (e.g., infrared) images, which may be used to provide information about the user 301 and the surrounding environment. The images captured by the camera can be analyzed to differentiate the user 301 from the surrounding environment. For example, the image analysis may include determining a profile or boundary of the user 301. The torso 302 can be based on the identified boundary and anatomical features of the user 301.

Identifying the torso 302 can be implemented in a variety of ways. In some cases, the torso 302 can include the user's 301 shoulders and a portion of the user's 301 torso such as an upper portion of the torso as illustrated in FIG. 3A. In other cases, the torso 302 can include a lower portion of the user's 301 torso such as an abdomen. The torso 302 may be identified based on image analysis that includes identifying specific anatomical features of a user and then defining the region of torso 302 based on these features. For example, the analysis can include identifying the upper boundary of the user's 301 shoulders and/or other prominent features such as an elbow or waist region of the user 301 and defining the torso 302 as the profile of the user 301 between the shoulder (e.g., upper boundary) and the elbow region (e.g., lower boundary). In some cases, the system can instruct the user to stand in a specific position or posture, and/or perform specific movements, which may be used to identify one or more anatomical features of the user 301.

Any suitable image analysis techniques may be used to identify the user. In some cases, information from an image (e.g., color information) can be used to differentiate between image data associated with the user 301 and image data associated with the user's surroundings. Additionally or alternatively, movement of the user 301 can be used to identify anatomical features of the user 301. For example, movement of the user 301 with respect to stationary background elements can be used to define a profile of the user 301 within the image data and/or determine anatomical features of a user.

The identified torso 302 can be used to define one or more sampling regions 304. For example, the sampling regions 304 can be an array of regions having a defined size that are positioned within the torso 302. One or more depth measurements can be taken within each sampling region and combined to produce measurement data for each of the sampling regions 304. In some cases, the depth measurements taken at each sampling region 304 can be averaged or otherwise combined to generate region data. The measurement data for each sampling region can be normalized, which can be based on the total depth changes within a region. The normalization may allow different regions to be compared, for example, because a central torso may have greater absolute movement than a peripheral chest region. For at least this reason, normalizing measurement data across different sampling regions may allow data for be collected for each region and data from different regions may be used to generate a respiration parameter. This region-based analysis may help provide robust analysis, during a sampling period, for example the region analysis may use data from different ones of the sampling regions 304 as the user 301 and/or the optical sensing unit moves and/or changes positions during the sampling period.

FIG. 3B shows an example of measurement data 350 collected from sampling region 304a during a sampling period. The measurement data 350 can include torso movement 306 which include depth changes over time. The measurement data 350 is shown as a graph that includes depth measurements 308 over time 310, for the sake of illustration. Although as implemented, this data may be process by a computer system, as described herein, and be stored and processed in computer readable formats such as binary data.

The measurement data 350 shows torso movement 306 for the sampling region 304a. In some cases, the measurement data 350 can include torso movement measurements for one or more of the other sampling regions 304. In some cases, the measurement data 350 can be analyzed in the frequency spectrum and one or more respiration parameters can be derived from the frequency-based analysis such as identifying a fundamental frequency using Fourier analysis.

In some cases, the measurement data 350 may not correspond to defined regions as illustrated in FIG. 3A. For example, the system may collect an array of data each corresponding to different locations across the torso 302. Each sampling point may be normalized and a depth map may be generated based on the combination of the sampling points. For example, each sensing location may correspond to a time-based depth signal. In some cases, this data may be used to determine how breathing movement propagates across the user's 301 torso 302. For example, movement propagation can be used to determine whether breathing movement is initiated in the upper chest region (e.g., chest area corresponding to the lungs) and/or whether and/or to what extent the user 301 is using their abdomen region during respiration. In some cases, different portions of a depth map can be compared to provide an indication of to what extent a user is using their chest versus their abdomen. In other cases, one or more sampling regions corresponding to a chest region of a user may be compared to one or more sampling regions corresponding to an abdomen of a user to determine to what extent a user is using their chest region versus their abdomen.

In some cases, machine learning can be applied to generate a parameterized torso model from images or other data collected by one or more cameras and/or sensors. Such a model may include latent variables such as identity parameters, pose parameters, and respiratory parameters, determining the overall body shape. As the user breathes, the respiratory parameters change accordingly in a cyclic fashion. Adherence metrics can also be estimated using these respiratory parameters.

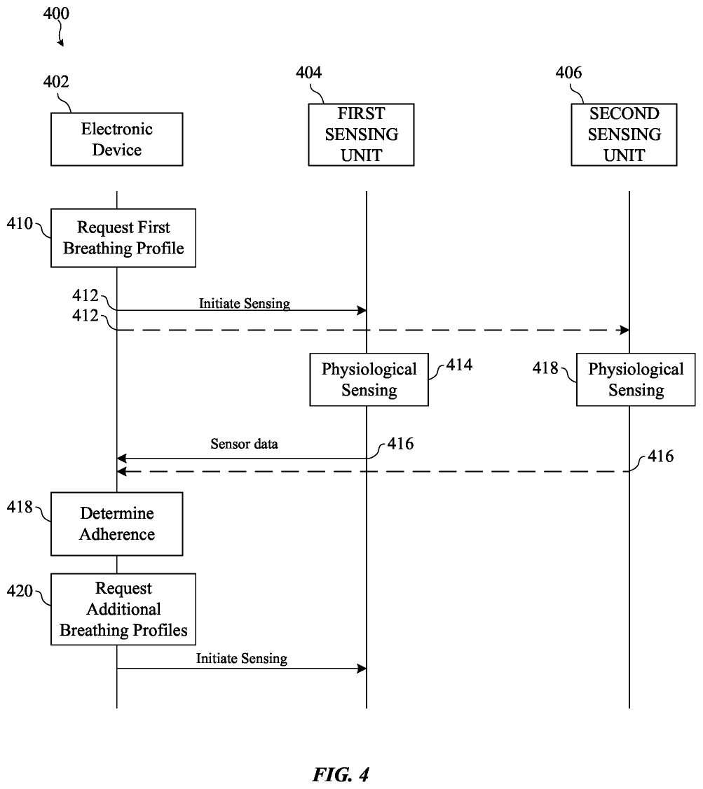

FIG. 4 shows an example signal flow 400 between different devices of a respiratory monitoring system, such as the respiratory monitoring systems described herein. The signal flow 400 can be between an electronic device 402, a first sensing unit 404, and a second sensing unit 406 that measure torso movement of a user. The electronic device 402 may be an example of the electronic devices described herein that have an output unit that outputs instructions for a user to perform guided breathing exercises as described herein. In some cases, the electronic device 402 can include the first sensing unit 404 and/or the second sensing unit 406. For example, the electronic device 402 can be a smartphone and include an optical sensing unit as described herein. In some cases, the second sensing unit 406 can be integrated into an electronic device that contacts a user and measures chest movements of the user as described herein. The electronic device 402 may correspond to device 108 and output unit 104 of the system described herein and with respect to FIG. 1. The first sensing unit 404 may correspond to the optical sensing unit 102 and the second sensing unit 406 may correspond to a second sensing unit such as a sensing unit of the electronic device as described herein and with respect to FIG. 1.

At 410, the electronic device 402 can output a request for a user to breathe according to a breathing profile as described herein. In response to outputting the request, the electronic device 402 can initiate a respiration measurement process at one or more of the first sensing unit 404 and the second sensing unit 406. In some cases, the respiration measurement process may include performing optical sensing of torso movements of a user. At 412, the electronic device may send one or more signals that initiate an optical sensing process at the first sensing unit 404. For example, the first sensing unit can identify a torso of a user and detect depth changes of the user's chest. In other cases, the respiration process can include performing a motion measurement process using the second sensing unit that is contacting the user. In these cases, at 412, the electronic device 402 may transmit one or more signals to the second sensing unit 406 to initiate a motion sensing process.

In some cases, the first sensing unit 404 and the second sensing unit 406 may operate together to perform respiration measurements for a user. For example, the first sensing unit 404 may track movement of the second sensing unit 406, which may correspond to movement of the user's chest. In some embodiments, the first and second sensing units 404, 406 can use wireless position sensing to determine chest movement. For example, each of the first and second sensing units 404, 406 can include one or more antennas and may transmit wireless signals (e.g., ultrawideband signals) between various ones of the antennas, which can be used to determine distance and/or positioning of the devices with respect to each other. This positioning data can be used to determine movement of a user's chest, such as changes in depth due to movement of the second sensing unit 406 with respect to the first sensing unit 404.

In other embodiments, the first sensing unit 404 can use the second sensing unit 406 as a visual target and can optically track the second sensing unit 406 to measure chest movement of a user. The second sensing unit can be an electronic device as described herein. In other cases, the second sensing unit 406 can be a device that does not include electronic components, but instead functions as an optical target for the first sensing unit 404. For example, the second sensing unit can be a device that couples to a chest of a user and provides an optical target for the first sensing unit 404.

In response to the electronic device sensing one or more signals to initiate physiological sensing at 412, the first sensing unit 404 can measure physiological parameters of a user at 414 and/or the second sensing unit 406 can measure physiological parameters of a user at 418. In cases where the first sensing unit 404 comprises an optical sensor, at 414, the first sensing unit may generate signals indicative of depth changes of the user's chest over a sampling period. Additionally or alternatively, the second sensing unit 406 may measure motion of user's torso using one or more motion sensors as described herein and generate signals indicative of the measured motion. The motion signals can include accelerometer outputs, gyroscope outputs, and/or other position sensing data.

In cases, where the first and second sensing units 404, 406 operate in coordination to track chest movements, the devices containing these sensing units may establish one or more communication channels that can be used to coordinate sensing activities and/or exchange sensing data. For example, when wireless signal positioning is used, the first sensing unit 404 may include one or more antennas that transmit wireless signals to the second sensing unit 406. The second sensing unit may include one or more antennas the receive the wireless signals, which can be any suitable radio-frequency signals including ultra-wide band (UWB) signals. Distances between different sets of antennas in the first and second sensing units 404, 406 can be determined based of signal transmission time, and the distance and/or position between the first and second sensing units 404, 406 can be determined from these signal transmissions. One or more of the electronic devices associated with the first and second sensing units 404 and 406 can determine depth changes based on the measuring distance and/or position changes between the sensing units.

At 416, the first sensing unit 404 and/or the second sensing unit 406 can transmit the measurement data to the electronic device 402. In some cases, the measurement data may include digitized signals that are indicative of the measured parameters and the electronic device 402 can process these signals to derive one or more respiration parameters for the sampling period. The signals can include time stamps or other data that can be used to correlate signals received from the first sensing unit 404 with the signals received from the second sensing unit 406. Accordingly, the measurement data from each of the sensing units can be compared and/or combined to determine respiration parameters for the user.

At 418, the electronic device can determine one or more adherence metrics for the user based on the measurement data received from the first and second sensing units 404 and 406. In some cases, the electronic device 402 may determine one or more adherence metrics using data from the first sensing unit 404 and independently determine one or more adherence metrics using data from the second sensing unit 406. The electronic device 402 may compare adherence metrics determined from the different sensing units to generate a combined metric. In other cases, one of the sensing units may be operated to generate primary adherence data and the other sensing unit can be operated to perform secondary adherence data that is used to supplement or update the primary adherence data. For example, the first sensing unit 404 can be operated to measure changes in chest depth of the user and the second sensing unit 406 can be operated to measure sources of noise such as breathing sounds of the user (e.g., sounds associated with inhale, exhale and breathe hold), transient chest movements such as due to coughing, and so on. In some cases, the second sensing unit 406 can measure secondary parameters such as breathing sounds, which can be analyzed with the primary measurements to determine one or more adherence metrics for the user.

At 420 the electronic device can determine whether to perform additional respiratory measurements. For example, the electronic device may output a request for a user to breathe according to a second breathing profile, and in response, can initiate a second sensing procedure at the first sensing unit 404 and the second sensing unit 406, which can be similar or the same as described with respect to the first sampling period at the first requested breathing profile. For example, the second breathing profile may be initiated in response to a user completing a first guided breathing exercise or a first portion of a guided breathing exercise. The system can determine one or more adherence metrics for the second breathing profile during a second guided breathing exercise.

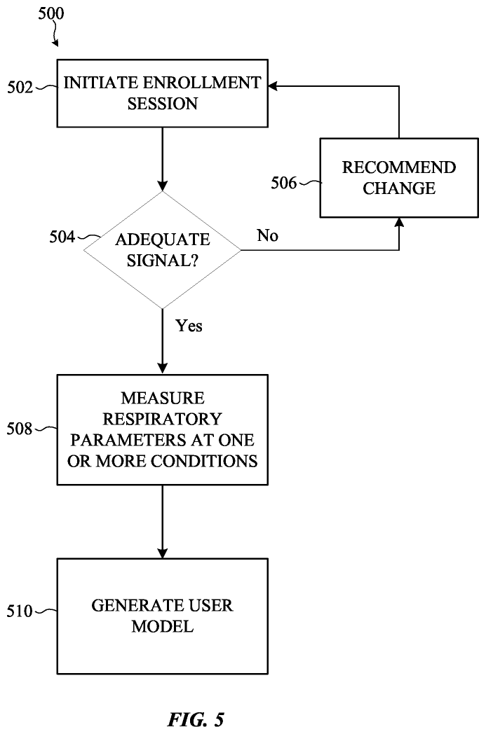

FIG. 5 shows an example process 500 for performing an enrollment session for respiratory sensing. The process can be performed by the respiratory sensing systems described herein such as a system 100. During the enrollment session, the system can collect and analyze data about the user that can be used as a baseline data set for the user. After the initial enrollment period, data collected by the system can be compared to the baseline data set.

At 502, the process 500 can include initiating an enrollment session for a user of the respiratory sensing system. The enrollment session may be performed to generate baseline respiratory data for a user which can be used to more accurately measure a user's breathing parameters, which may help increase an accuracy of the determined adherence metrics. In some cases, the enrollment session can be activated the first time that a user initiates respiratory sensing function of the electronic device. For example, the enrollment session may be activated in response to a user opening an application for respiratory sensing. In some cases, initiating an enrollment session can include collecting data about a user. The system may collect physiological parameters for a user. The enrollment session can be performed for a defined amount of time or until stable baseline data sets are developed for the user. For example, determining a stable baseline data set can include determining values or ranges for specific physiological parameters within a defined confidence interval.

In some cases, the enrollment session includes measuring breathing parameters using traditional respiratory measurement techniques including spirometry measurements, which can be compared to measured chest movement data obtained by the systems described herein, used to calibrate chest movement measurement by the systems described herein, and/or otherwise used in the respiratory measurement analysis. In some cases, the system (e.g., the output unit or other electronic device) can perform guided breathing for spirometry measurements or other forced exhalation techniques. For example, the electronic device can output video, images, audio, haptic cues to guide the user through a forced exhalation protocol such as a spirometry measurement.

At 504, the process 500 can include determining whether adequate respiratory measurements can be achieved. For example, the ability to get an adequate signal strength, signal quality and/or repeatability over a sampling period may be dependent on a variety of environmental factors. These can include the positioning of the sensing unit(s) with respect to the user, movement of the sensing unit(s) and/or the user, background noise, and so on. In cases where the sensing unit includes an optical sensor, lighting, the clothing being worn be the user, user and/or camera movement, and/or the like can all affect the signal strength and/or quality. Accordingly, at 504 the process 500 may evaluate one or more conditions to determine whether adequate respiratory measurements can be achieved to accurately determine one or more adherence metrics to a requested breathing profile.

In some cases, the system can evaluate the positioning of the sensing unit(s) with respect to the user, which may include determining whether a torso of a user is within a field of view of an optical sensor, the signal strength of one or more sensors, signal noise, movement of the sensors and/or user, and so on. In cases where an optical sensing unit is being used, the system may analyze a user's clothing to determine whether it can measure a user's chest movement. For example, if a user's clothing is sized or positioned to not be in contact with the user's torso, the system may not be able to detect chest movement of the user through the clothing.

At 506, the process 500 can include recommending changes to the user to improve the respiratory measurements by the system. For example, in the cases where a user's clothing sufficiently masks chest movement of the user from optical depth measurements, the system may prompt the user to adjust or change clothing or use a different sensing modality such as a movement sensor that is coupled to the user's chest. In other cases, the system may instruct the user to change the positioning of a sensing unit, such as an electronic device that has a camera, and/or instruct the user to change position and/or posture. In some cases, the system can provide live feedback on a display or using other outputs (e.g., audio instructions) for the user, for example, where to move the electronic device or how to reposition themselves within a field of view of the camera. In some instances, the system may notify a user that the adherence functionality with be unavailable during a guided breathing session.

At 508, the process can include measuring breathing parameters of a user to generate a baseline data set for the user. This can include measuring breathing parameters at one or more different conditions. For example, the system may measure a user's natural breathing patterns by instructing the user to position themselves in front on the optical sensing unit and breathe naturally for a period of time. The system may perform multiple of these natural breathing sessions to develop a resting or natural breathing profile for the user. Additionally or alternatively, the system may instruct the user to breathe according to one or more breathing profiles, which may include faster or slower breathing rates, one or more breathing patterns and/or the like. The system can measure physiological parameters of the user at each of the different breathing rates, which can include measuring chest movement, heart rate, the user's actual breathing rate, oxygen saturation, temperature, breathing sounds and/or other physiological parameters. In some cases, the system may evaluate a user's ability to match a requested breathing profile, whether a user is within a defined range of the requested profile, consistently faster, consistently slower, and so on.

In some cases, the during the enrollment session, the system can correlate measurements from the sensing unit(s) to other measurement modalities. For example, the process 500 can include taking spirometry measurements during the enrollment session. The spirometry measurement data can be correlated to data measured by the respiratory sensing system, which can include correlating a forced vital capacity (FVC), forced expiratory volume (FEV) and peak expiratory flow (PEF) to the chest measurement data from the respiratory sensing system. In some cases, chest motion data measured by a first sensing modality, such as optical depth sensing, can be correlated with physiological data measured by a second sensing device such as a wearable health monitoring device. For example, the second sensing device can include a smartwatch that measures heart rate, ECG data, oxygen saturation, and/or the like.

Additionally or alternatively, the system can analyze the effect of different user positions/postures on the user's breathing motion. For example, the system may instruct the user to perform different breathing sessions in different positions such as sitting, standing, laying down, from a front view, from a back view, and/or at different postures such as standing upright versus hunched over. This data may be integrated into the baseline data set such that the user can collect respiratory motion data in a variety of different positions/postures and the baseline data can be used to normalize these differences. In other cases, the system may instruct the user to perform a breathing exercise in a specific posture to increase the accuracy, repeatability and/or to otherwise improve the breathing measurements.

At 510, the process 500 can include generating a user model (e.g., parameterized model) based on the data collected during the enrollment session. The user model may be part of the baseline data set and include three-dimensional model data for a user. For example, the system may generate three-dimensional model data for a user's torso which can be based on sensor data and/or data input from a user. The sensor data and/or user inputs can include parameters related to a user's height, weight, sex, and/or the like. In some cases, this data can be used to derive and/or generate model data such as a body mass index, a body surface area, dimensions/proportions of a user's torso, lung capacity, and/or the like. The user model can be used to refine the respiratory analysis, for example to help determine how user specific factors such as the user's body shape, lung capacity and so on affect a user's breathing movements. In some cases, the user model can be updated over time. For example, as respiratory sensing sessions are performed, data from these sessions can be used to update and/or track changes in the user model.

In some case, machine learning can be applied to generate a 3D parameterized torso model. Such a model may include latent variables such as identity parameters, pose parameters, and respiratory parameters, determining the overall body shape. As the user breathes, the respiratory parameters change accordingly in a cyclic fashion. These parameters can be subsequently used to provide adherence metrics during the use of the system.

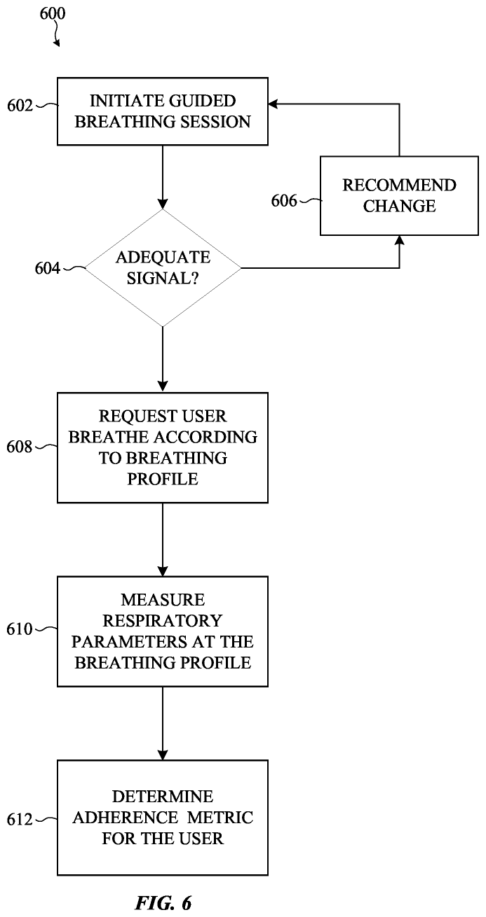

FIG. 6 shows an example process 600 for performing a respiratory sensing session for a user. The process can be performed by the respiratory sensing systems described herein such as a system 100.

At 602, the process 600 can include initiating a guided breathing session. A guided breathing session can be initiated in a variety of ways. In some cases, the guided breathing session can be initiated by a user. For example, an electronic device can include an application for performing respiratory measurements and the application can include controls for a user to initiate a guided breathing session.

At 604, the process 600 can include determining whether an adequate respiratory measurement can be achieved, which can be the same or similar to operation 504 of process 500. Evaluating the signal strength and/or quality can be performed prior to each sensing session and/or during the sensing session. In some cases, this can include evaluating a user's clothing, posture, and positioning with respect to the sensing unit(s), and/or the like as described herein. If at 604, the system determines that an adequate signal cannot be achieved the system may make one or more recommendations to the user at 606 to help improve the measurement signals, which can be similar to operation 506 of process 500. For example, the system can instruct the user to change clothes, move to reposition themselves within a field of view of a sensing unit, move the electronic device containing the sensing unit and so on.

At 608, the process 600 can include requesting a user to breathe according to a breathing profile. The breathing profile can define one or more breathing parameters that the user is intended to perform. For example, the system can provide outputs that instruct the user to breathe at a defined breathing rate (e.g., a defined number of breaths per minute), at a defined breathing depth (e.g., defined in terms of a user's maximum inhale and exhale capacity), and/or other suitable breathing parameters. In some cases, the outputs can include a breathing pattern for the user to match based on the breathing profile. For example, the breathing pattern can include a box breathing technique (e.g., four-square breathing), diaphragmatic breathing (e.g., belly breathing), pursed lip breathing, 4-7-8 breathing, and/or any other suitable breathing technique and the system can output cues (e.g., visual cues, audio cues, haptic cues, and so on) that indicate the breathing pattern to the user.

In some cases, the request to breathing at the breathing profile can include one or more audio outputs, visual outputs, haptic outputs and/or other suitable outputs as described herein. In some cases, the breathing profile can define one or more breathing parameters which may be used to determine an adherence metric as described herein. For example, the one or more breathing parameters may include a breathing rate, a breathing depth, and so on. In some cases, the breathing parameters may be defined as a time-varying signal that defines target torso movements over time. For example, the time-varying signal can include inhale timing, exhale timing, target depth changes of the chest during the inhale motion, the exhale motion and so on. Additionally the time varying signal can define a hold between inhale and exhale and/or between exhale and inhale (e.g., four-square breathing).

At 610, the process 600 can include measuring breathing parameters of the user while the user is being requested to breath according to the breathing profile. The system can define one or more sampling regions along a torso of a user. The system can use a sensing unit such as an optical sensing unit to measure changes in depth along the one or more regions. In some cases, the system can select at least one sampling region and determine a breathing parameter using the measured depth changes at the selected sampling region(s).

In some cases, the system can sense other breathing related parameters such as breathing sounds, images of a user's mouth and/or nose, and/or other suitable parameters. For example, the images of the user's mouth and/or nose may be used to determine extents to which the user is breathing through their mouth and nose. In some cases, the breathing profile may include parameters that cause the system to instruct breathing in specific conditions such as mouth open, mouth closed, inhaling through the nose and exhaling through the mouth and so on. The system may use cameras to determine if the user is and/or to what extent a user is breathing through their mouth or nose and provide feedback to the user based on the measured data. Additionally or alternatively, the system may track other user parameters such as eye motion, which may be used to determine if a user is focused on a guided breathing exercise (e.g., are the user's eyes open or closed) or if they are distracted (e.g., eyes focused on other objects in the environment).

The breathing parameters may provide an indication of to what extent the user inhaled and exhaled during the breathing profile. The respiratory sensing system may determine one or more additional or alternative breathing parameters based on the measured depth changes during the first sampling period which can include determining peak-to-peak amplitude of the user's chest movement, absolute amplitude, the morphology of the torso movement over the sampling period, and so on.

At 612, the process 600 can include determining an adherence metric for the requested breathing profile. The adherence metric can indicate a correspondence between movement of the torso and the breathing profile. The adherence metric can be generated based on comparing metrics from derived from movement of the torso to target metrics for a requested breathing profile. For example, if the breathing profile defines particular breathing timing (e.g., inhale for a first amount of time, hold for a second among of time, exhale for a third amount of time, and so on), the movement data from measuring the torso can be used to determine the user's breathing timing and comparing the user's breathing timing to the defined metrics of the breathing profile. The adherence metric can be a correspondence between the torso movement of the user (and/or other measured breathing parameters such as breathing sounds) with one or more characteristics of the breathing profile.

The adherence metric can indicate how the breathing cycle-timing of the user (e.g., inhale and exhale timing) matches the cycle timing of the requested breathing profile. This can include determining phase shifts between the user's measured breathing cycle and the requested breathing profile. Additional or alternatively, the adherence metric can indicate how the depth changes in the user's torso matches the breathing depth changes of the requested breathing profile. For example, the breathing profile may request inhale and exhale depths based on the maximum inhale and exhale capacity of the user, which may be determined during the enrollment period or other breathing session. Accordingly, the adherence metric may indicate to what extent the user is matching the requested depth changes (e.g., depth at maximum exhale, depth at maximum inhale, and so on).

In some cases, the system can change or update the breathing profile based on the adherence metric satisfying one or more criteria. For example, if the adherence metric indicates that the user is lagging behind the breathing rate of the requested breathing profile by a defined amount and/or for a defined duration (e.g., number of breathing cycles), the system may update the breathing profile to include a slower breathing rate. The system may change other parameters of the breathing profile accordingly. For example, if the user is performing a box breathing technique, the system may update the breathing profile to decrease each of the inhale time, the inhale hold time, the exhale time and the exhale hold time. In other cases, the system can update the breathing profile to change an inhale depth, exhale depth, breathing pattern or any other suitable parameter.

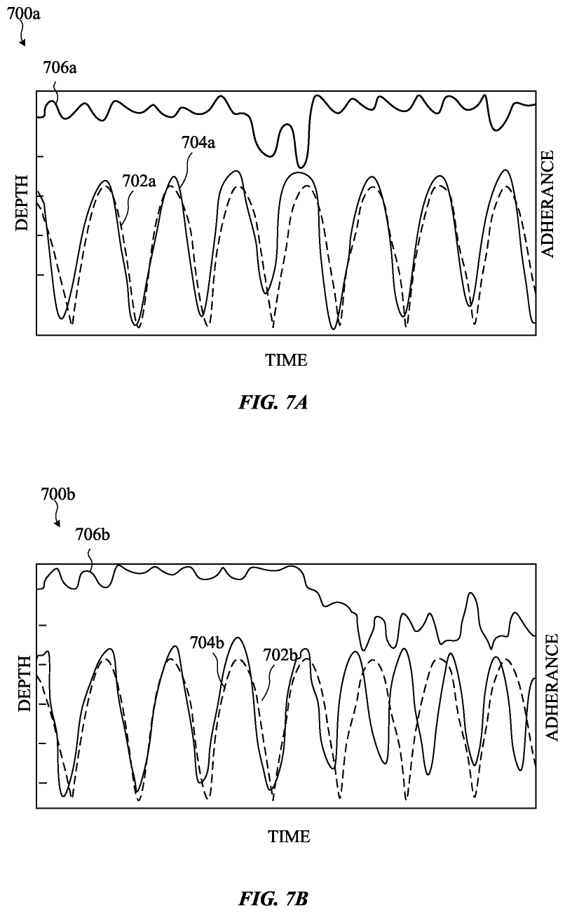

FIGS. 7A-7B show an example of torso movement data measured for a requested breathing profile and example adherence data determined from the measured torso movement data. The torso movement data can include chest depth measurements taken over the respective sampling period, which include depth changes over time. The torso movement data and requested breathing profile are shown on a graph that includes depth measurements over time, for the sake of illustration. Although as implemented, this data may be processed by a computer system, as described herein, and be stored and processed in computer readable formats such as binary data.

FIG. 7A shows an example of a requested breathing profile data 702a and torso depth measurements 704a taken during a sampling period 700a. The example shown in FIG. 7A show torso depth measurements 704a that have close correspondence to the requested breathing profile. The breathing profile data 702a can indicate the requested changes in depth over time. For example, the requested breathing profile 702a includes a constant breathing rate and defined inhale and exhale depths. The torso depth measurements 704a can be measured changes in the user's chest depth. In some cases, the system can determine an adherence data set 706a which illustrates the correspondence between the torso depth measurements 704a and the requested breathing profile 702a. The adherence data can be generated based on comparing metrics from derived from torso depth measurements 704a to target metrics for the requested breathing profile 702a. For example, if the requested breathing profile 702a defines particular breathing timing (e.g., inhale for a first amount of time, hold for a second among of time, exhale for a third amount of time, and so on), the torso depth measurements 704a can be used to determine the user's breathing timing and comparing the user's breathing timing to the defined metrics of the breathing profile 702a. The adherence metric can be a correspondence between the torso movement of the user (and/or other measured breathing parameters such as breathing sounds) with one or more characteristics of the breathing profile.

In some cases, the system may determine one adherence metrics from the adherence data set such as an average adherence, a maximum and/or minimum adherence, and/or any other suitable metric. Additionally or alternatively, the system may output the one or more adherence metrics, the torso depth measurements 704a, and/or the requested breathing profile to the user in real-time or after the breathing secession has been completed.

FIG. 7B shows another example of a requested breathing profile data 702b and chest depth measurements 704b taken during a sampling period 700b. The example shown in FIG. 7B shows chest depth measurements 704b that initially have close correspondence to the requested breathing profile during a first portion of the sampling period 700b and lower correspondence during a second portion of the sampling period 700b. The breathing profile data 702b can indicate the requested changes in breathing depth over time. The system can determine an adherence data set 706b which illustrates the correspondence between the chest depth measurements 704b and the requested breathing profile 702b. As shown in FIG. 7B, the transition in the adherence data set 706b part way through the sampling period can indicate a drop in the adherence between the chest depth measurements 704b and the requested breathing profile.

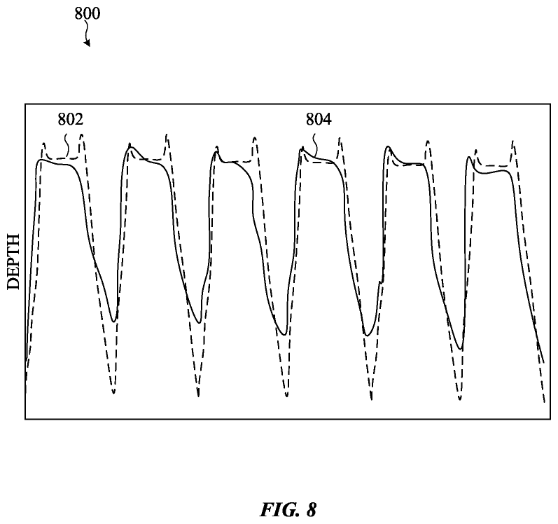

FIG. 8 shows an example of time-varying breathing profiles 802 and chest depth measurements 804 for the time-varying breathing profiles 802. The chest movement data can include chest depth measurements taken over the respective sampling period, which include depth changes over time. The chest movement data and requested breathing profile are shown on a graph that includes depth measurements over time, for the sake of illustration. Although as implemented, this data may be processed by a computer system, as described herein, and be stored and processed in computer readable formats such as binary data.

The requested time-varying breathing profile 802 can include a breathing pattern which includes a breath hold between inhalation and exhalation. The torso depth measurements 804 for a user can be used to determine adherence data, which indicates a correspondence between the requested time-varying breathing profile 802 and user's torso movements. In the example shown in FIG. 8, the adherence data may be used to generate one or more adherence metrics. For example, an adherence metric can indicate an overall (e.g., average) adherence for the sampling period 800. Another adherence metric may indicate the user's ability to match the breathing rate and a different adherence metric may indicate the user's ability to match the breathing depth. Additionally or alternatively, another breathing metric can indicate the user's ability to perform the breath hold. Accordingly, the adherence data determined from the requested time-varying breathing profile 802 and the torso depth measurements 804 can be used to derive one or more adherence metrics to provide information about how the user is matching the requested breathing profile.

The requested breathing profile 802 is an example of a breathing profile that can be requested by the system. The breathing profiles can define various breathing parameters which may include breathing patterns such as a box breathing technique (e.g., four-square breathing), diaphragmatic breathing (e.g., belly breathing), pursed lip breathing, 4-7-8 breathing, and/or any other suitable breathing technique. In other examples, the breathing profile may define breathing parameters such as breathing at a specified breathing rate without specifying a breathing depth and vice versa. In these cases, the adherence metric may indicate a user's ability to match the requested breathing profile. For example, the adherence metric may indicate the user's ability to match a breathing rate. In some cases, the system may use other measurements techniques, such as recording breathing sounds of the user, to determine or assist in determining the user's breathing rate and thereby in determining the adherence metric.

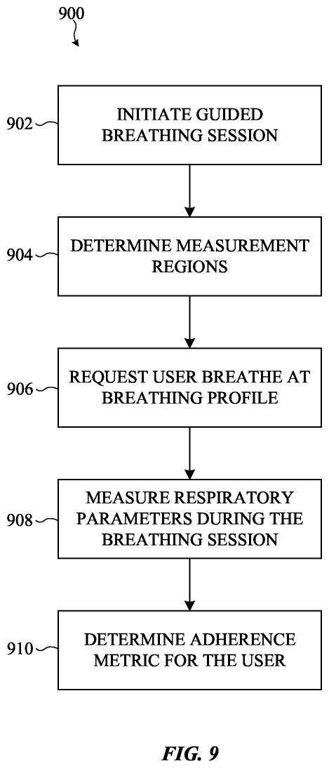

FIG. 9 shows an example process 900 for performing a guided breathing session for a user. The process 900 can be performed by the respiratory sensing systems described herein such as a system 100.

At 902, the process 900 can include initiating a guided breathing session. A guided breathing session can be initiated in a variety of ways. In some cases, the guided breathing session can be initiated by a user. For example, an electronic device can include an application for performing respiratory measurements and the application can include controls for a user to initiate a guided breathing session.

At 904, the process 900 can include determining multiple regions for measuring depth changes of a use's torso, shoulder movement, head movement, arm movements and/or measuring other breathing parameters such as a user's mouth shape. In some cases, the system may be configured to analyze a user's chest depth measurements and a user's belly depth measurements, which may be used to compare how much the user is using their chest and their belly to breathe. The system can be configured to define one or more first measurement regions along a chest portion of a user as described herein. Additionally, the system can be configured to define one or more second measurement regions along a belly portion of the user, as described herein. Accordingly, as a user is performing a breathing exercise, the system can measure both changes in chest depth of the user and belly depth of the user. In some cases, the system can determine one or more adherence metrics for a first sensing region such as a chest region of a user, and one or more adherence metrics for a second sensing region such as a belly region of a user. Some breathing profiles may cause outputs instructing a user to primarily breathe using belly movement and maintaining their chest stationary. Accordingly, the different adherence metrics for the chest and belly may indicate how well a user is able to match this type of breathing profile.

Additionally or alternatively, the system may be configured to measure other breathing paraments such as a user's mouth movements, mouth shape (e.g., open area), nostril shape and so on. In these cases, the system (e.g., optical sensing system) may define one or more sampling regions that includes the user's mouth and/or nose. The system may collect images and/or measure the user's mouth movements during a requested breathing profile. For example, a requested breathing profile may include breathing parameters related to a user's mouth shape (e.g., target shape, percent of max open area, and so on) and one or more adherence metrics can be determined based on how measure parameters of a user's mouth shape match the defined parameters for the breathing profile.

At 906, the process 900 can include requesting a user to breathe according to a breathing profile. The breathing profile can define one or more breathing parameters that the user is intended to perform, as described herein. In some cases, the breathing profile may define breathing parameters such as chest verses belly breathing. A breathing profile can define a breathing pattern for the user to match, as described herein. The request to breathe at the breathing profile can include one or more audio outputs, visual outputs, haptic outputs and/or other suitable outputs as described herein.

The breathing profile can define one or more breathing parameters which may be used to determine an adherence metric as described herein. For example, the one or more breathing parameter may include a breathing rate, a breathing depth, type of breath (e.g., is a given breath considered a chest breath or a belly breath based on defined metrics), and so on. In some cases, the breathing parameters may be defined as a time-varying signal that defines target chest movements and/or belly movements over time. For example, the time-varying signal can include inhale timing, exhale timing, target depth changes of the chest during the inhale motion, the exhale motion and so on. Additionally the time varying signal can define a hold between inhale and exhale and/or between exhale and inhale (e.g., four-square breathing).

In some cases, the requested breathing profile may include parameters such as a mouth shape (e.g., open mouth, pursed lips and/or the like), for the user to breath using through their nose and/or through their mouth (e.g., inhale through nose and exhale through mouth) or any other suitable parameters.

At 908, the process 900 can include measuring breathing parameters of the user while they are breathing at the requested breathing profile. The system can measure breathing parameters at the one or more measurement regions. The system can use a sensing unit such as an optical sensing unit to measure changes in depth along the one or more regions. Additionally or alternatively, the system may use an optical sensing unit to measure mouth shape as described herein. In some cases, the system can sense other breathing related parameters such as breathing sounds, images of a user's mouth and/or nose, and/or other suitable parameters. For example, the images of the user's mouth and/or nose may be used to determine extents to which the user is breathing through their mouth and nose.

The breathing parameters may provide an indication of to what extent the user inhaled and exhaled during the breathing profile. For example, the measured breathing parameter can include depth measurement for the chest, depth measurements for the belly, or depth measurements for other defined sensing regions.

At 910, the process 900 can include determining one or more adherence metrics for the requested breathing profile. The adherence metric can indicate a correspondence between movement of the torso and the breathing profile, as described herein. For example, the adherence metric can indicate how closely a user is matching requested chest and/or belly movements.

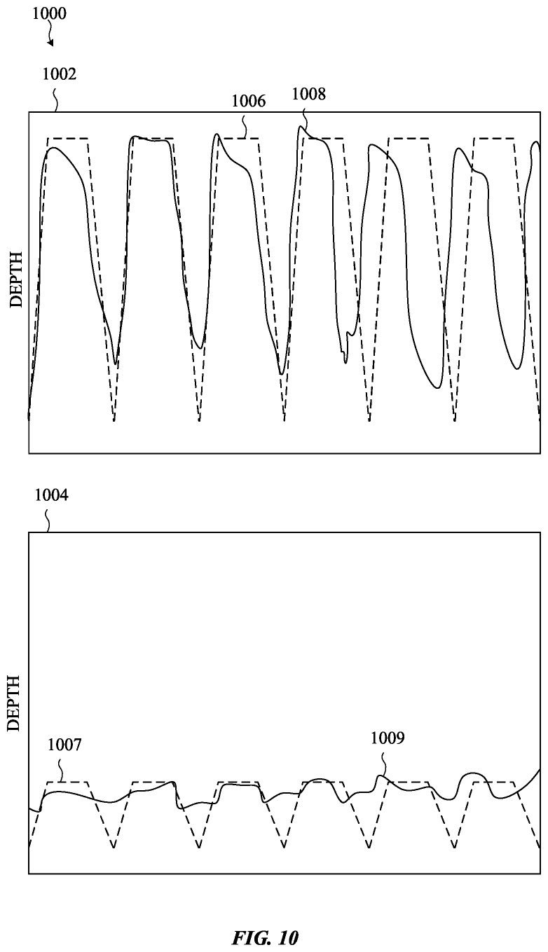

FIG. 10 shows an example of torso movement data 1000 for multiple regions of the torso during a guided breathing exercise. The torso movement data 1000 can include changes in depth for an abdominal sensing region 1002 and changes in depth for a chest sensing region 1004. The breathing profile may include multiple sensing regions and define different parameters for each region. For example, the breathing profile may include an abdominal sensing region 1002 that defines requested abdominal movement 1006 for the user to match. The breathing profile may also include chest sensing region 1004 that defines the chest movement 1007 for the user to match. The system may collect abdominal depth measurements 1008 and chest depth measurements 1009 using the techniques described herein. In some cases, the system can generate a combined adherence metric that includes both abdominal and chest movements. In other cases, different adherence metrics can be determined for different sensing regions for example, the system can generate a first adherence metric indicating a correspondence between movement of the user's chest and the requested second breathing profile 1007 and a second adherence metric indicating a correspondence between movement of the user's abdomen and the requested first breathing profile 1006.

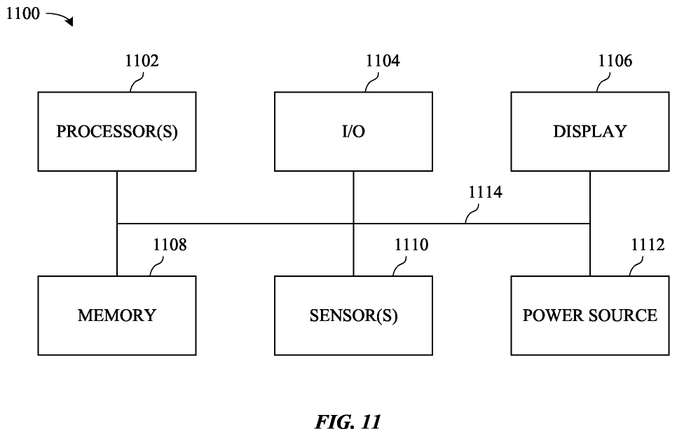

FIG. 11 is an example block diagram of a respiratory monitoring system 1100, which can take the form of any of the devices as described with references to FIGS. 1-10. The respiratory monitoring system 1100 can include a processor 1102, an input/output (I/O) mechanism 1104 (e.g., wired or wireless communications interfaces), a display 1106, memory 1108, sensors 1110 (e.g., physiological sensors such as those described herein), and a power source 1112 (e.g., a rechargeable battery). The processor 1102 can control some or all of the operations of the respiratory monitoring system 1100. The processor 1102 can communicate, either directly or indirectly, with some or all of the components of the respiratory monitoring system 1100. For example, a system bus or other communication mechanism 1114 can provide communication between the processor 1102, the I/O mechanism 1104, the memory 1108, the sensors 1110, and the power source 1112.

The processor 1102 can be implemented as any electronic device capable of processing, receiving, or transmitting data or instructions. For example, the processor 1102 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements. The processing unit can be programmed to perform the various aspects of the systems described herein.

It should be noted that the components of the respiratory monitoring system 1100 can be controlled by multiple processors. For example, select components of the respiratory monitoring system 1100 (e.g., a sensor 1110) may be controlled by a first processor and other components of the respiratory monitoring system 1100 (e.g., the I/O 1104) may be controlled by a second processor, where the first and second processors may or may not be in communication with each other.

The I/O device 1104 can transmit and/or receive data from a user or another electronic device. An I/O device can transmit electronic signals via a communications network, such as a wireless and/or wired network connection. Examples of wireless and wired network connections include, but are not limited to, cellular, Wi-Fi, Bluetooth, IR, and Ethernet connections. In some cases, the I/O device 1104 can communicate with an external electronic device, such as a smartphone, electronic device, or other portable electronic device, as described here.

The respiratory monitoring system may optionally include a display 1106 such as a liquid-crystal display (LCD), an organic light emitting diode (OLED) display, a light emitting diode (LED) display, or the like. If the display 1106 is an LCD, the display 1106 may also include a backlight component that can be controlled to provide variable levels of display brightness. If the display 1106 is an OLED or LED type display, the brightness of the display 1106 may be controlled by modifying the electrical signals that are provided to display elements. The display 1106 may correspond to any of the displays shown or described herein.

The memory 1108 can store electronic data that can be used by the respiratory monitoring system 1100. For example, the memory 1108 can store electrical data or content such as, for example, audio and video files, documents and applications, device settings and user preferences, timing signals, control signals, and data structures or databases. The memory 1108 can be configured as any type of memory. By way of example only, the memory 1108 can be implemented as random access memory, read-only memory, Flash memory, removable memory, other types of storage elements, or combinations of such devices.

The respiratory monitoring system 1100 may also include one or more sensors 1110 positioned almost anywhere on the respiratory monitoring system 1100. The sensor(s) 1110 can be configured to sense one or more types of parameters, such as but not limited to, pressure, light, touch, heat, movement, relative motion, biometric data (e.g., biological parameters), and so on. For example, the sensor(s) 1110 may include a heat sensor, a position sensor, a light or optical sensor, an accelerometer, a pressure transducer, a gyroscope, a magnetometer, a health monitoring sensor, and so on. Additionally, the one or more sensors 1110 can utilize any suitable sensing technology, including, but not limited to, capacitive, ultrasonic, resistive, optical, ultrasound, piezoelectric, and thermal sensing technology.

The power source 1112 can be implemented with any device capable of providing energy to the respiratory monitoring system 1100. For example, the power source 1112 may be one or more batteries or rechargeable batteries. Additionally or alternatively, the power source 1112 can be a power connector or power cord that connects the respiratory monitoring system 1100 to another power source, such as a wall outlet.

As described above, one aspect of the present technology is monitoring and managing physiological conditions of a user such respiratory movements and the like. The present disclosure contemplates that in some instances this gathered data may include personal information data that uniquely identifies or can be used to contact or locate a specific person. Such personal information data can include demographic data, location-based data, telephone numbers, email addresses, Twitter IDs (or other social media aliases or handles), home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to provide haptic or audiovisual outputs that are tailored to the user. Further, other uses for personal information data that benefit the user are also contemplated by the present disclosure. For instance, health and fitness data may be used to provide insights into a user's general wellness or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining the privacy and security of personal information data. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and revised to adhere to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining spatial parameters, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, haptic outputs may be provided based on non-personal information data or a bare minimum amount of personal information, such as events or states at the device associated with a user, other non-personal information, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A system for analyzing a breathing parameter of a user, the system comprising:
an optical sensing unit configured to:
detect movement of a torso of the user; and
output one or more signals indicative of the movement of the torso;
an electronic device configured to request for the user to breathe according to a breathing profile during a time period; and
a processing unit configured to:
receive the one or more signals output by the optical sensing unit during the time period;
determine an adherence metric using the one or more signals and the breathing profile, the adherence metric indicating a correspondence between the movement of the torso and the breathing profile requested during the time period.

2. The system of claim 1, wherein:
detecting the movement of the torso comprises detecting changes in depth of the torso with respect to the optical sensing unit;
the breathing profile defines a depth parameter; and
determining the adherence metric comprises determining differences between the depth parameter and the changes in depth of the torso.

3. The system of claim 1, wherein:
detecting the movement of the torso comprises detecting breathing cycle timing of the user;
the breathing profile defines a timing parameter; and
determining the adherence metric comprises determining differences between the timing parameter and breathing cycle timing of the user.

4. The system of claim 1, wherein the processing unit is configured to:
determine an updated breathing profile based on the adherence metric satisfying one or more criteria;
cause the electronic device to request the user to breathe according to the updated breathing profile during a second time period; and
determine an updated adherence metric for the second time period.

5. The system of claim 4, wherein determining the updated breathing profile comprises adjusting one or more breathing parameters of the breathing profile.

6. The system of claim 1, wherein the request for the user to breathe according to the breathing profile comprises an audio output indicating a breathing parameter of the requested breathing profile.

7. The system of claim 1, wherein the breathing profile comprises a breathing pattern.

8. The system of claim 1, wherein:
the request for the user to breathe according to the breathing profile comprises a visual output indicating a breathing pattern, a breathing depth or both; and
the visual pattern includes an indication of the movement of the torso.

9. A system for analyzing user adherence to a requested breathing profile, the system comprising:
a sensing unit configured to:
detect movement of a torso of a user; and
output one or more signals indicative of the detected movement of the torso;
an electronic device configured to provide a request for a user to breathe according to a breathing profile during a time period; and
a processing unit programmed to:
receive the one or more signals;
determine an adherence metric using the one or more signals and the breathing profile.

10. The system of claim 9 wherein:
the one or more signals include:
one or more first signals indicative of detected movement of a first region of the; and
one or more second signals indicative of detected movement of a second region of the torso; and
the processing unit is configured to determine the adherence metric using the one or more first signals and the one or more second signals.

11. The system of claim 10, wherein the adherence metric comprises:
a first metric corresponding to the movement of the first region; and
a second metric corresponding to the movement of the second region.

12. The system of claim 9, wherein the sensing unit comprises an optical sensing unit configured to measures changes in depth between the torso and the sensing unit.

13. The system of claim 9, wherein:
the sensing unit comprises an optical sensing unit configured to measure a mouth shape of the user; and
the adherence metric is determined using the measured mouth shape of the user.

14. The system of claim 9, wherein the request for the user to breath according to the breathing profile comprises an audio output.

15. The system of claim 9, wherein the request for the user to breath according to the breathing profile comprises a visual output.

16. A method for measuring a breathing parameter of a user, the method comprising:
outputting, from an electronic device, a request for the user to breath in accordance with a breathing profile during a time period;
obtaining, by an optical sensing unit, a set of respiratory measurements during the time period, the set of respiratory measurements comprising measurements corresponding to movement of a torso of the user; and
determining, by a processing unit, an adherence metric for the user using the set of respiratory measurements and the breathing profile, the adherence metric indicating a correspondence between movement of the torso and the breathing profile requested during the time period.

17. The method of claim 16 wherein obtaining the set of respiratory measurements comprises detecting changes in depth of the torso with respect to the optical sensing unit.

18. The method of claim 17 wherein:
the breathing profile defines a depth metric; and
determining the adherence metric comprises determining differences between the depth metric and the changes in depth of the torso.

19. The method of claim 16, wherein obtaining the set of respiratory measurements comprises detecting breathing cycle timing of the user.

20. The method of claim 19, wherein:
the breathing profile defines a timing metric; and
determining the adherence metric comprises determining differences between the timing metric and breathing cycle timing of the user.
